(19) United States
(12) Patent Application Publication
(10) Pub. No.: US 2016/0220241 A1
(43) Pub. Date: Aug. 4, 2016

(54) SUTURE COLLAR FOR USE WITH AN INTRODUCER DURING DIRECT AORTIC PROCEDURES

(71) Applicant: Medtronic Vascular Galway, Ballybrit (IE)

(72) Inventors: Sean Gorman, Ballybrit (IE); Niall Duffy, Ballybrit (IE)

(21) Appl. No.: 14/614,063

(22) Filed: Feb. 4, 2015

(51) Int. Cl. A61B 17/04; A61F 2/24

(52) U.S. Cl. CPC A61B 17/0401; A61F 2/2427; A61B 2017/0445

(57) ABSTRACT

A suture collar configured for use with an introducer sheath. The suture collar includes an outer ring component defining an opening there-though and an inner ring component concentrically disposed within the opening of the outer ring component. The outer ring component is configured to be sutured to a target surface of a patient. The inner ring component defines an opening there-through that is configured to receive the introducer sheath. In a first configuration of the suture collar, a longitudinal axis of the inner ring component is aligned with a longitudinal axis of the outer ring component. The inner ring component is configured to move relative to the outer ring component such that in a second configuration of the suture collar the longitudinal axis of the inner ring component is angularly offset with the longitudinal axis of the outer ring component.

### FIELD OF THE INVENTION

The invention relates to introducer assemblies for use during direct aortic procedures, and more particularly, relates to suture collars for use with an introducer during direct aortic procedures.

### BACKGROUND OF THE INVENTION

Currently, the replacement of a deficient cardiac valve is often performed by opening the thorax, placing the patient under extracorporeal circulation, temporarily stopping the heart, surgically opening the heart, excising the deficient valve, and then implanting a prosthetic valve in its place. This procedure generally requires prolonged patient hospitalization, as well as extensive and often painful recovery.

Recently, minimally invasive approaches have been developed to facilitate catheter-based implantation of valve prostheses in the beating heart, intending to obviate the need for the use of classical sternotomy and cardiopulmonary bypass. For example, U.S. Pat. No. 8,016,877 to Seguin et al. illustrates a technique and a device for replacing a deficient heart valve by percutaneous route. An expandable prosthetic valve is compressed about a catheter, inserted inside a lumen within the body, such as the femoral artery, and delivered to a desired location in the heart. Additionally, U.S. Pat. No. 7,914,569 to Nguyen et al. discloses advancing the catheter containing the prosthesis in a retrograde manner through the femoral artery and into the descending aorta, over the aortic arch, through the ascending aorta and inside the defective aortic valve. This procedure can be assisted by fluoroscopic guidance. Once the position of the catheter containing the prosthesis is confirmed, a sheath containing the prosthesis can be moved proximally, allowing the valve prosthesis to self-expand. Alternatively, a balloon catheter may be used to expand the valve prosthesis.

However, percutaneous delivery routes require peripheral arterial access and cannulation. In some patients, these routes are contraindicated or have an increased risk of vascular complications due to the presence of small vessel size, severe atherosclerosis, tortuosity and/or calcification. For patients with no suitable femoral or axillary access, a direct aortic approach may be utilized to deliver a transcatheter prosthetic heart valve. A direct aortic approach may be carried out via a mini-thoracotomy. An introducer is positioned directly onto the exterior surface of the aorta, and a prosthetic valve implantation is performed by advancing a standard delivery system through the introducer. Direct aortic valve implantation includes special advantages of high control of valve deployment with no adverse effect on the left ventricle structure or function.

Proper positioning and alignment of the delivery system is essential to a successful prosthetic valve deployment. Thus, when the standard delivery system is advanced through the introducer, it may be necessary to selectively steer or angle the delivery system toward the target implantation site, thereby resulting in pulling or stretching the aorta which may cause damage thereto. Embodiments hereof relate to a component that permits selective tilting of the introducer and delivery system during a direct aortic procedure.

### BRIEF SUMMARY OF THE INVENTION

Embodiments hereof relate to a suture collar configured for use with an introducer sheath. The suture collar includes an outer ring component defining an opening there-though and an inner ring component concentrically disposed within the opening of the outer ring component. The outer ring component is configured to be sutured to a target surface of a patient. The inner ring component defines an opening there-through that is configured to receive the introducer sheath. In a first configuration of the suture collar, a longitudinal axis of the inner ring component is aligned with a longitudinal axis of the outer ring component. The inner ring component is configured to move relative to the outer ring component such that in a second configuration of the suture collar the longitudinal axis of the inner ring component is angularly offset with the longitudinal axis of the outer ring component.

Embodiments hereof also relate to an introducer assembly including an introducer sheath and a suture collar configured to be positioned over the introducer sheath. The suture collar includes an outer ring component defining an opening there-though and an inner ring component concentrically disposed within the opening of the outer ring component. The outer ring component is configured to be sutured to a target surface of a patient and an inner surface of the outer ring component is concave. The inner ring component defines an opening there-through that is configured to receive the introducer sheath. In a first configuration of the suture collar, a longitudinal axis of the inner ring component is aligned with a longitudinal axis of the outer ring component. An outer surface of the inner ring component is convex and is configured to slidingly mate with the concave inner surface of the outer ring component to permit the inner ring component and the introducer sheath received therein to move relative to the outer ring component such that in a second configuration of the suture collar the longitudinal axes of the inner ring component and the introducer sheath received therein are angularly offset with the longitudinal axis of the outer ring component.

Embodiments hereof also relate to an introducer assembly including an introducer sheath and a suture collar configured to be positioned over the introducer sheath. The suture collar includes an outer ring component defining an opening there-though and an inner ring component concentrically disposed within the opening of the outer ring component. The outer ring component is configured to be sutured to a target surface of a patient. The inner ring component defines an opening there-through that is configured to receive the introducer sheath. In a first configuration of the suture collar, a longitudinal axis of the inner ring component is aligned with a longitudinal axis of the outer ring component. A membrane radially extends between the outer and inner ring components, wherein the membrane is formed from an elastic material to permit the inner ring component and the introducer sheath received therein to move relative to the outer ring component such that in a second configuration of the suture collar the longitudinal axes of the inner ring component and the introducer sheath received therein are angularly offset with the longitudinal axis of the outer ring component.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a perspective view of a suture collar according to an embodiment hereof, wherein the suture collar includes an inner ring component that is configured to tilt or pivot relative to an outer ring component.

FIG. 2 is a top end view of the suture collar of FIG. 1.

FIG. 2A is a cross-sectional view taken along line A-A of FIG. 2.

FIG. 3 is a bottom end view of a suture collar according to another embodiment hereof, wherein the suture collar includes a plurality of suture holes for receiving one or more purse string sutures to attach the suture collar to an outer surface of a body vessel such as an aorta.

FIG. 4 is a side view of the suture collar of FIG. 1 disposed over an introducer sheath of an introducer.

FIG. 5 is a cross-sectional view of the suture collar of FIG. 1 with the introducer sheath of the introducer of FIG. 4 shown in phantom, wherein the suture collar is in a first configuration in which a longitudinal axis of the inner ring component, as well as a longitudinal axis of the introducer sheath of the introducer, is aligned with a longitudinal axis of the outer ring component.

FIG. 6 is a cross-sectional view of the suture collar of FIG. 1 with the introducer sheath of the introducer of FIG. 4 shown in phantom, wherein the suture collar is in a second configuration in which the longitudinal axis of the inner ring component, as well as the longitudinal axis of the introducer sheath of the introducer is angularly offset with the longitudinal axis of the outer ring component.

FIG. 7 is an illustration of the suture collar of FIG. 1 and the introducer of FIG. 4 being utilized for a direct aortic procedure within the aortic arch.

FIG. 8 is a top view of a suture collar according to another embodiment hereof, wherein the suture collar includes an inner ring component that is configured to tilt or pivot relative to an outer ring component via a flexible membrane extending between the inner and outer ring components.

FIG. 8A is a cross-sectional view taken along line A-A of FIG. 8.

FIG. 9 is a side view of an adjustable suture collar according to another embodiment hereof disposed over an introducer sheath of an introducer, wherein the adjustable suture collar is shown in a first longitudinal position along the introducer sheath.

FIG. 10 is a side view of the adjustable suture collar of FIG. 9 disposed over the introducer sheath of the introducer, wherein the adjustable suture collar is shown in a second longitudinal position along the introducer sheath.

FIG. 11 is a sectional perspective exploded view of the adjustable suture collar of FIG. 9.

FIG. 12 is a sectional perspective exploded view of an adjustable suture collar according to another embodiment hereof.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the aorta, the invention may also be used in any other body passageways where it is deemed useful. Furthermore, there is no intention to be bound by any expressed or implied theory presented in the preceding technical field, background, brief summary or the following detailed description.

Embodiments hereof relate to suture collars configured for use with an introducer during a direct aortic procedure. Suture collars according to embodiments hereof are configured to be positioned over an introducer sheath and are configured to abut against, and optionally be sutured to, an outer surface of the aorta during a direct aortic procedure. An inner ring component of the suture collar, as well as the introducer sheath received there-through, is configured to tilt or pivot relative to a stationary outer ring component of the suture collar so that the introducer sheath may be selectively steered or angled toward a target implantation site. Further, a delivery system positioned through the introducer sheath may be selectively steered or angled toward a target implantation site without pulling or stretching the aorta to which the suture collar is attached as will be explained in more detail herein.

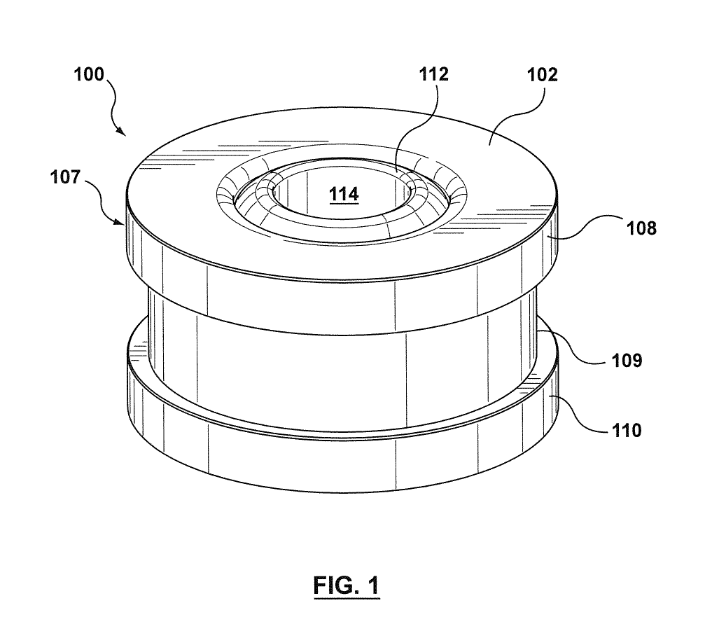

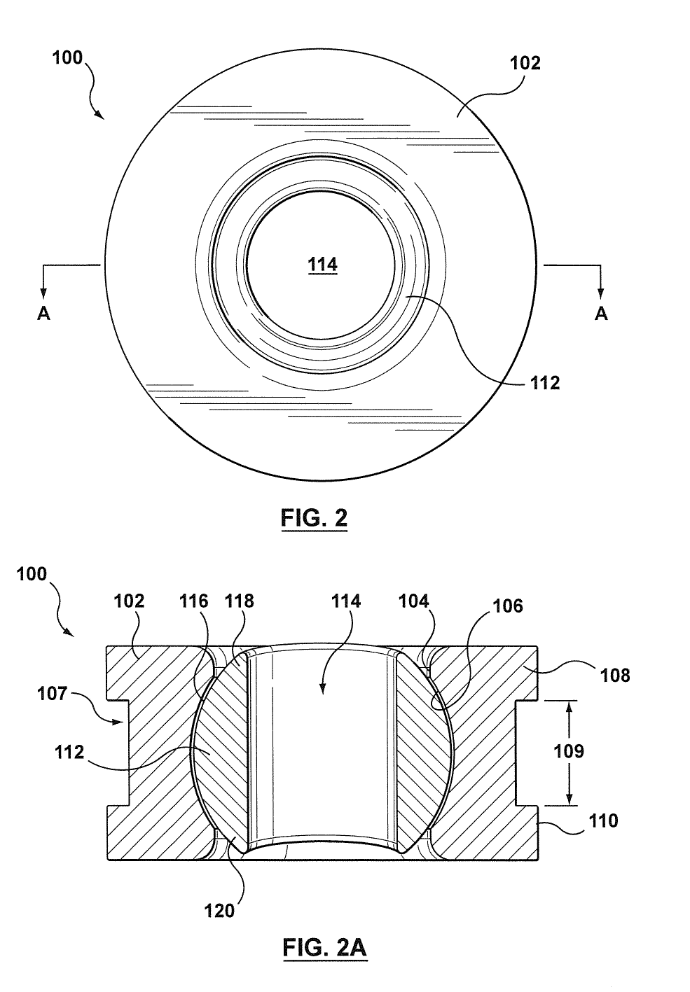

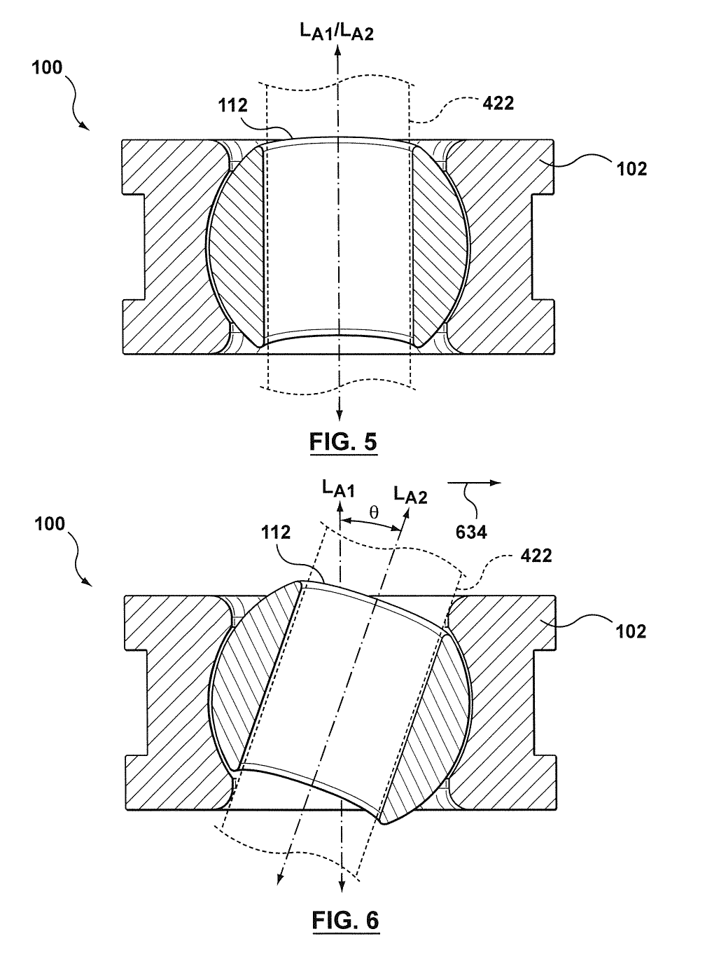

With reference to FIGS. 1-2A, suture collar 100 includes an outer ring component 102 defining an opening 104 there-though and an inner ring component 112 concentrically disposed within opening 104 of the outer ring component. FIG. 1 is a perspective view of suture collar 100, FIG. 2 is a top view of suture collar 100, and FIG. 2A is a cross-sectional view of suture collar 100 taken along line A-A of FIG. 2. Outer ring component 102 is configured to be sutured to a target surface of a patient as will be explained in more detail herein. Inner ring component 112 defines an opening 114 there-through that is configured to receive an introducer sheath (not shown in FIGS. 1-2A). In a first configuration of suture collar 100, as shown and described below with respect to FIG. 5, a longitudinal axis of inner ring component 112 is aligned with a longitudinal axis of outer ring component 102. Inner ring component 112 is configured to move relative to outer ring component 10 such that in a second configuration of suture collar 100, as shown and described below with respect to FIG. 6, the longitudinal axis of inner ring component 112 is angularly offset with the longitudinal axis of outer ring component 102.

More particularly, outer ring component 102 is an annular or ring element having opening 104 there-though. An inner surface 106 of outer ring component 102 is concave or curved in a first direction, and is sized to house a spherical or ball-shaped element. An outer surface 107 of outer ring component 102 includes a first annular ridge or flange 108 at a first end or edge thereof and a second annular ridge or flange 110 at a second or opposing end or edge thereof. An intermediate portion 109 is formed between first and second flanges 108, 110 and may be considered a groove or channel between first and second flanges 108, 110. Each flange 108, 110 extends radially outward relative to intermediate portion 109 of outer ring component 102 such that the outer diameter of the outer ring component at each flange is greater than the outer diameter of the outer ring component at intermediate portion 109. Each flange 108, 110 also extends around the full circumference of outer ring component 102. In an embodiment, one or more purse string sutures (not shown) wrap or circle around intermediate portion 109 to attach or sew suture collar 100 to an outer surface of a body vessel such as an aorta. Flanges 108, 110 thus define a region, i.e., intermediate portion 109, that receives one or more purse string sutures for attachment to the outer surface of a body vessel and prevents the sutures from slipping off suture collar 100. Although separately described, it will be understood by one of ordinary skill in the art that first and second flanges 108, 110 are integrally formed on outer ring component 102.

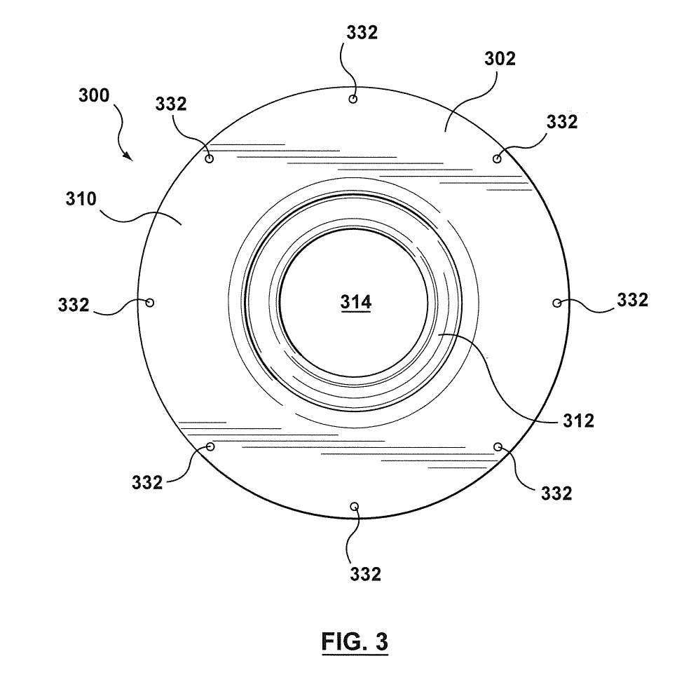

In another embodiment, rather than tying one or more sutures around intermediate portion 109 of suture collar 100, the suture collar may additionally and/or alternatively include suture holes for receiving one or more purse string sutures for attachment to the outer surface of a body vessel. More particularly, as shown in the embodiment of FIG. 3, which is a bottom view of a suture collar 300, a second annular ridge or flange 310 includes a plurality of suture holes 332 there-through for receiving one or more purse string sutures (not shown) to attach or sew suture collar 300 to an outer surface of a body vessel such as an aorta. Suture collar 300 includes an outer ring component 302 and an inner ring component 312 which defines an opening 314. Suture collar 300 is similar in structure to suture collar 100 except that second annular flange 310 may optionally extend further radially outward than flange 110 and a first or top flange such as flange 108 may optionally be omitted. When second annular ridge or flange 310 is placed in contact with the outside of the aorta, purse string sutures can be positioned through suture holes 332 to secure outer ring component 302 to the aorta. Although shown with eight suture holes 332 equally spaced around flange 310, it will be understood by one of ordinary skill in the art that the number of suture holes 332 may vary according to application.

Inner ring component 112 is housed within outer ring component 102. Inner ring component 112 is an annular or ring element having opening 114 there-through that is configured to receive an introducer sheath (not shown in FIGS. 1-2A). Opening 114 of inner ring component 112 is sized to snugly receive the introducer sheath. Stated another way, the introducer sheath is disposed within inner ring component 112 such that there is sufficient space or room there-between for the introducer sheath to be moved or slidable in a longitudinal direction relative to inner ring component 112. In other words, there is not a tight interference or friction fit between the introducer sheath and inner ring component 112. However, the introducer sheath is disposed within inner ring component 112 such that inner ring component 112 tilts or pivots with tilting or pivoting of the introducer sheath. In other words, when the introducer sheath (or a catheter or delivery system disposed within the introducer sheath) is tilted or pivoted by a clinician, the introducer sheath contacts an inner surface of inner ring component 112 and thereby tilts or pivots inner ring component 112. The two components may move simultaneously as an ensemble structure, or there may be a slight delay in the rotation of inner ring component 112 by the introducer sheath.

An outer surface 116 of inner ring component 112 is convex or curved in a second direction that opposes the first direction of concave outer surface 106. As such, convex outer surface 116 of inner ring component 112 is configured to slidingly mate or correspond to concave inner surface 106 of outer ring component 102. Stated another way, inner ring component 112 may be considered a spherical or ball-shaped element having a first truncated end 118 and a second or opposing truncated end 120. Similar to a sphere or ball, inner ring component 112 rolls, revolves, spins, swivels, or otherwise moves within outer ring component 102 due to the mating convex and concave surfaces.

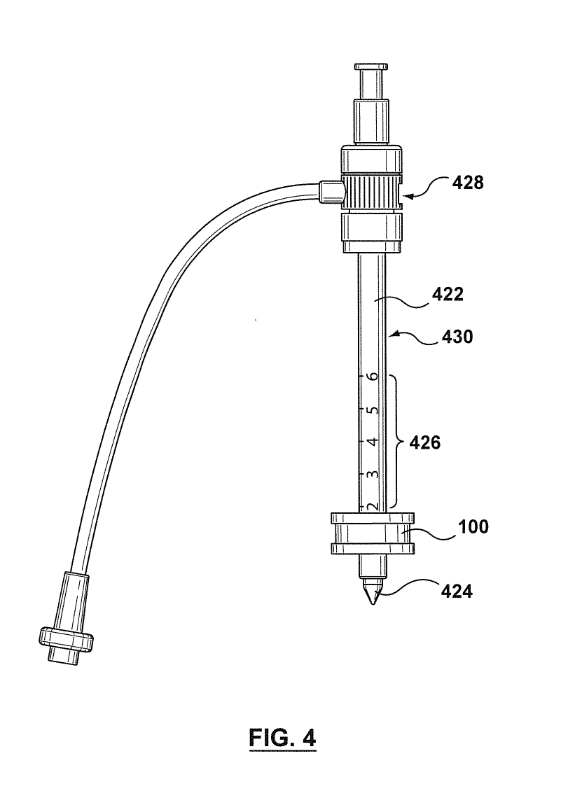

As shown in FIG. 4, opening 114 of inner ring component 112 is configured to receive an introducer sheath 422 of an introducer 430 as described above such that suture collar 100 is configured to be positioned over the introducer sheath. Suture collar 100 is typically formed as a separate component that is placed or positioned over introducer sheath 422 prior to a procedure. Stated another way, suture collar 100 is removable from introducer sheath 422. For example, suture collar 100 and introducer 430 are manufactured as separate or distinct components that are shipped and/or stored together and then assembled into an introducer assembly prior to the procedure. As will be understood by those of ordinary skill in the art, introducer 430 is merely exemplary and suture collar 100 may be utilized with other introducers known in the art. In this embodiment, introducer 430 includes introducer sheath 422 which is a tubular component sized to slidingly receive a delivery system such as a prosthetic valve delivery system. Introducer sheath 422 also includes a depth gauge 426, which as the name implies, measures the depth that the introducer sheath extends beyond a fixed reference point, such as the exterior surface of the aorta, into a patient's body. A hub 428 is attached to a proximal end of introducer sheath 422. A hemostatic valve (not shown) is positioned within hub 428 and is configured to permit passage of a delivery system or catheter into introducer sheath 422 while preventing blood leakage as will be understood by one of ordinary skill in the art.

As shown in FIG. 4, a removable dilator 424 extends through introducer sheath 422. Dilator 424 is a separate or independent component from introducer 430, and is removable from introducer 430. Dilator 424 is shown in FIG. 4 as extending from a distal end of introducer 430. Dilator 424 is configured to expand or enlarge an incision that is made on a body vessel such as an aorta when introducer 430 is being positioned in the body vessel. The length of dilator 424 varies according to application, and a relatively short dilator is utilized when suture collar 100 and introducer 430 are used on the aorta. Dilator 424 extends the entire length of introducer sheath 422, and is proximally retracted or removed once introducer 430 is positioned in the body vessel.

Referring now to FIG. 5, which is a cross-sectional view of suture collar 100 with introducer sheath 422 shown in phantom, suture collar 100 is shown in its first configuration, i.e., when in a nominal or normal configuration in which no force is applied thereto. In the first or nominal configuration of suture collar 100, a longitudinal axis L_{A2} of inner ring component 112 is aligned with or overlaps a longitudinal axis L_{A1} of outer ring component 102. Since introducer sheath 422 is disposed or positioned through opening 114 of inner ring component 112, the longitudinal axis of the introducer sheath corresponds to longitudinal axis L_{A2} of inner ring component 112. Stated another way, when assembled together, introducer sheath 422 and inner ring component 112 have the same longitudinal axis, i.e., longitudinal axis L_{A2}.

FIG. 6 is a cross-sectional view of suture collar 100 with introducer sheath 422 shown in phantom, with suture collar 100 shown in a second or tilted configuration in which the longitudinal axis L_{A2} of inner ring component 112 (and introducer sheath 422) is angularly offset with longitudinal axis L_{A1} of outer ring component 102. As described above, inner ring component 112 rolls, revolves, spins, swivels, or otherwise moves in any direction within outer ring component 102 due to the mating convex and concave surfaces 116, 106, respectively. Inner ring component 112 and introducer sheath 422 received therein tilt or pivot relative to outer ring component 102, which is fixed or stationary since it is sutured or secured to the exterior surface of the aorta. In FIG. 6, the longitudinal axis L_{A2} of inner ring component 112 (and introducer sheath 422) is shown to be angularly offset with longitudinal axis L_{A1} of outer ring component 102 by a tilting angle θ. Tilting angle θ as shown is only exemplary because tilting angle θ is a user-controlled variable. Stated another way, a user controls the amount or degree that inner ring component 112 (and introducer sheath 422) is titled relative to outer ring component 102. In an embodiment, tilting angle θ is limited due to the contact between introducer sheath 422 and outer ring component 102 and may range between 0 and 45 degrees. In addition, although FIG. 6 illustrates inner ring component 112 (and introducer sheath 422) being tilted in a first tilting direction indicated by directional arrow 634, such a tilting direction is exemplary because the tilting direction is also a user-controlled variable. Stated another way, a user controls which direction inner ring component 112 (and introducer sheath 422) is tilted relative to outer ring component 102. With respect to the tilting direction, inner ring component 112 (and introducer sheath 422) is configured to be tilted in any direction relative to outer ring component 102.

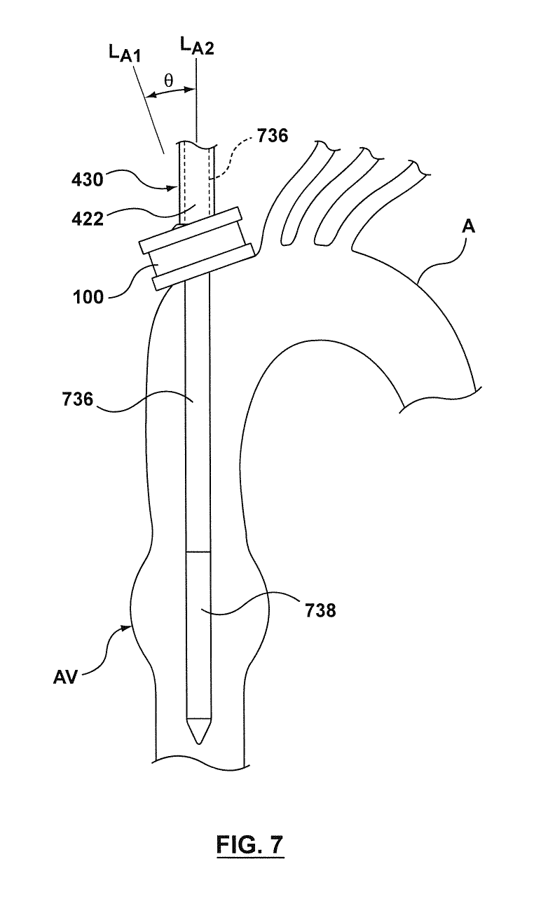

During use, inner ring component 112 (and introducer sheath 422) is configured to be selectively steered or angled toward a target implantation site. For example, FIG. 7 illustrates suture collar 100 attached to an exterior surface of an aorta A near the aortic arch. Introducer sheath 422 is shown positioned through suture collar 100. Further, a prosthetic valve delivery system 736 is shown positioned through introducer sheath 422. Introducer sheath 422 and prosthetic valve delivery system 736 may be selectively steered or angled toward a target implantation site, such as a native aortic valve AV, without pulling or stretching the aorta A to which suture collar 100 is attached.

More particularly, as previously described, suture collar 100 and introducer 430 are manufactured as separate or distinct components that are shipped and/or stored together and then assembled prior to the procedure. Prior to use, suture collar 100 is positioned over introducer sheath 422. Generally, a mini-thoracotomy is made, the pericardium is opened to expose the aorta, and an incision is made on the aorta A. The aorta, as defined herein, can include the exterior surface and lumen of the descending aorta, aortic arch, ascending aorta and aortic sinus. A purse string suture procedure may be performed prior to making an incision. After an incision is made on the aorta A, introducer 430 is passed through the incision using dilator 424 to dilate the incision, with suture collar 100 positioned over or assembled onto the introducer sheath 422. As introducer 430 is positioned through the incision, the surgeon can use the purse string sutures to minimize bleeding and then the purse string sutures are tied to suture collar 100 to hold it in place via intermediate portion 109 of suture collar 100 as described above, and/or alternatively via a plurality of suture holes 332 as described above. Thus, outer ring component 102 of suture collar 100 is secured to the exterior surface of the aorta A via one or more purse string sutures. Tying the purse string suture(s) from the aorta around suture collar 100, or through suture holes of the suture collar, maintains a haemostatic seal as well as controls the position of introducer sheath 422 while the physician is performing the direct aortic procedure. Stated another way, suture collar 100 is coupled to and sealed against the aorta A via the purse string sutures and thus suture collar 100 prevents blood from exiting the body. In addition, the hemostatic valve of hub 428 (not shown in FIG. 7) of introducer 430 also prevents blood from exiting the body.

Once suture collar 100 and introducer 430 are positioned as desired, a prosthetic valve delivery system 736 is advanced through introducer 430. Delivery system 736 may be any delivery system known in the art for percutaneously delivering a prosthesis, such as but not limited to a heart valve prosthesis. Delivery system 736 includes a radially collapsed heart valve prosthesis (not shown in FIG. 7) that is loaded into a capsule 738 at a distal end of the delivery system. If necessary, in order to direct delivery system 736 to the target implantation site of the native aortic valve AV, introducer sheath 422 and prosthetic valve delivery system 736 may be selectively steered or angled toward the native aortic valve AV. More particularly, as described above, the inner ring component of suture collar 100 and introducer sheath 422 (having longitudinal axis L_{A2} shown in FIG. 7) are configured to be tilted or moved relative to the outer ring component of suture collar 100 (having longitudinal axis L_{A1} shown in FIG. 7). Since the outer ring component of suture collar 100 remains stationary and sutured to the aorta A, the inner ring component of suture collar 100 and introducer sheath 422 are configured to be moved relative to the outer ring component of suture collar 100 without pulling or stretching the aorta A. Thus, advantageously, prosthetic valve delivery system 736 that is advanced through introducer 430 may be selectively steered or angled toward the native aortic valve AV without pulling or stretching the aorta A. Proper positioning and alignment of prosthetic valve delivery system 726 is essential to a successful prosthetic valve deployment.

After the inner ring component of suture collar 100 and introducer sheath 422 are tilted relative to the outer ring component of suture collar 100 as desired in order to direct prosthetic valve delivery system 736 toward the native aortic valve AV, the delivery system is advanced in a retrograde manner within the aorta A until distal capsule 738 reaches the desired implantation location within the native aortic valve AV. The location of distal capsule 738 can be checked by medical imaging. Once in the proper position, distal capsule 738 is refracted in the proximal direction, thereby uncovering the heart valve prosthesis contained in distal capsule 738. The prosthesis can be self-expanding or can be mechanically expanded, such as by balloon inflation. After the prosthesis is fully deployed, prosthetic valve delivery system 736 can be removed from the aorta A. Suture collar 100 can be detached from the aorta A, and the incision can be closed by sutures.

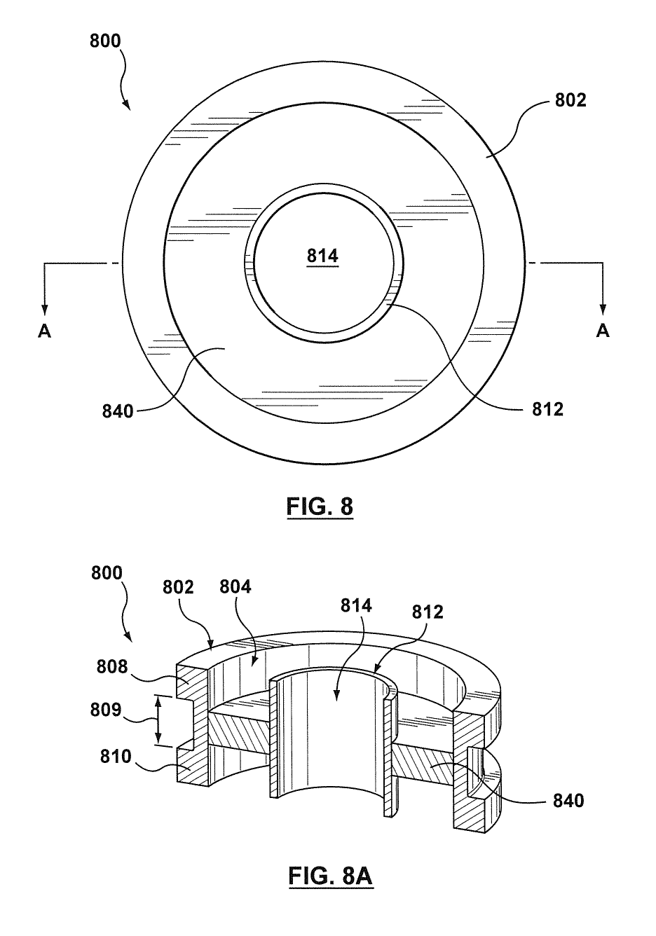

In another embodiment hereof, rather than mating convex and concave surfaces, the suture collar includes a flexible membrane that is configured to permit an inner ring component to tilt or pivot relative to an outer ring component. More particularly, FIGS. 8-8A illustrate a suture collar 800 according to another embodiment hereof. FIG. 8 is a top view of suture collar 800, while FIG. 8A is a cross-sectional view taken along line A-A of FIG. 8. Outer ring component 802 is similar to outer ring component 102. More particularly, outer ring component 802 is an annular or ring element having opening 804 there-though. Further, outer ring component 802 includes a first annular ridge or flange 808 at a first end or edge thereof and a second annular ridge or flange 810 at a second or opposing end or edge thereof. Each flange 808, 810 extends radially outward from outer surface 807 of outer ring component 802 such that the outer diameter of the outer ring component at each flange is greater than the outer diameter of the outer ring component at intermediate portion 809. Each flange 808, 810 also extends around the full circumference of outer ring component 102.

Further, inner ring component 812 is similar to inner ring component 112. More particularly, inner ring component 812 is an annular or ring element having opening 814 there-through that is configured to receive an introducer sheath (not shown in FIGS. 8-8A). In this embodiment, however, a flexible membrane 840 radially extends between outer and inner ring components 802, 812. Flexible membrane 840 is a planar component that extends approximately through a center or mid-line plane of outer and inner ring components 802, 812. Flexible membrane 840 is biased to position suture collar 800 in its first configuration, i.e., a nominal or normal configuration in which a longitudinal axis of inner ring component 812 is aligned with or overlaps a longitudinal axis of outer ring component 802. Flexible membrane 840 is formed from an elastic material to permit inner ring component 812 to tilt or pivot relative to outer ring component 802. Stated another way, the elastic material of membrane 840 allows suture collar 800 to assume its second or tilted configuration in which the longitudinal axis of inner ring component 812 (and introducer sheath received therein) is angularly offset with the longitudinal axis of outer ring component 802. Inner ring component 812 and the introducer sheath received therein tilt or pivot relative to outer ring component 802, which is fixed or stationary since it is sutured or secured to the exterior surface of the aorta. When no force is applied thereto, i.e., when a user does not apply any force to the inner ring component and/or introducer sheath received there-through, the elastic material of flexible membrane 840 causes suture collar 800 to resume its first configuration in which inner ring component 812 is longitudinally aligned within outer ring component 802. As such, flexible membrane 840 is configured to be self-centering due to the elastic material thereof. Suitable materials for flexible membrane 840 include but are not limited to silicone rubber, a low-grade PEBAX, a low grade polyurethane, and other polymeric materials having elastic characteristics. Flexible membrane 840 may be molded or over-molded into position between outer and inner ring components 802, 812.

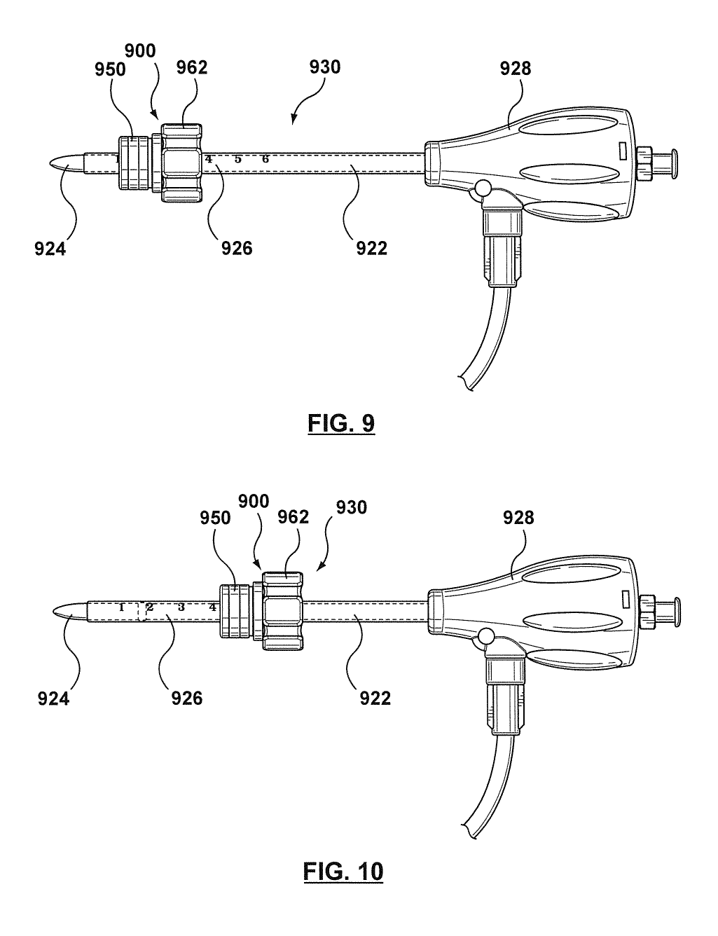

In another embodiment hereof shown in FIGS. 9-11, rather than being configured to permit selectively tilting of an introducer sheath received there-through, an adjustable suture collar 900 is configured to be slidingly disposed over an introducer sheath 922 of an introducer 930 and is configured to be selectively tightened or locked into place. Introducer 930 is similar to introducer 130 and, in addition to introducer sheath 922, a depth gauge 926, and a hemostatic valve 928. A dilator 924 is shown extending through introducer 930. Suture collar 900 includes a base ring component 950 and a removable ring component 962. In a first configuration, in which removable ring component 962 is removed or loosely disposed over base ring component 950, suture collar 900 is configured to slide or move freely over the outer surface of introducer sheath 922 such that suture collar 900 is configured to be selectively positioned at a desired longitudinal position along depth gauge 926. In a second configuration, in which removable ring component 962 is tightly disposed over or locked onto base ring component 950, suture collar 900 is tightened or locked onto the outer surface of introducer sheath 922 at the desired longitudinal position along depth gauge 926 and is not able to slide or move freely over the outer surface of introducer sheath 922. FIG. 9 is a side view of suture collar 900 in its second configuration, locked on the outer surface of introducer sheath 922 at a first longitudinal position along introducer sheath 922, and FIG. 10 is a side view of suture collar 900 in its second configuration, locked on the outer surface of introducer sheath 922 at a second longitudinal position along introducer sheath 922. When transitioning between the longitudinal positions of FIG. 9 and FIG. 10, suture collar 900 is in its first configuration.

More particularly, FIG. 11 is a sectional perspective exploded view of adjustable suture collar 900. Base ring component 950 of suture collar 900 defines an opening 952 there-through that is configured to receive an introducer sheath (not shown in FIG. 11). Base ring component 950 has a first longitudinal portion 954 having an outer surface 956 with a plurality of threads 958 formed thereon and a second longitudinal portion 960. Second longitudinal portion 960 includes a first annular ridge or flange 908 at a first end or edge thereof and a second annular ridge or flange 910 at a second or opposing end or edge thereof. Each flange 908, 910 extends radially outward such that the outer diameter of the outer ring component at each flange is greater than the outer diameter of the outer ring component at an intermediate portion 909. Each flange 908, 910 also extends around the full circumference of outer ring component 902.

Removable ring component 962 of suture collar 900 defines an opening 963 there-through and has an inner surface 964 with a plurality of grooves 966 that are configured to mate with the plurality of threads 958 on first longitudinal portion 954 of base ring component 950. A diameter or size of inner surface 964 tapers or decreases from a first end 967 to a second end 968, and is sized such that at least a portion of inner surface 964, i.e., second end 968, has a smaller diameter than an outer diameter of first longitudinal portion 954 of base ring component 950. First longitudinal portion 954 of base ring component 950 is formed from a flexible, compressible material, while second longitudinal portion 960 of base ring component 950 and removable ring component 962 are both formed from a relatively rigid, non-compressible material. The flexible material of first longitudinal portion 954 is an elastomeric material that is molded or over-molded onto second longitudinal portion 960 of base ring component 950.

In order to be tightened or locked onto the outer surface of the introducer sheath, i.e., in order to change or transform suture collar 900 from its first configuration into its second configuration, removable ring component 962 is screwed onto base ring component 950 via mating grooves 966 and threads 958 of removable ring component 962 and base ring component 950, respectively. When removable ring component 962 is screwed onto base ring component 950 and removable ring component 962 is concentrically disposed over first longitudinal portion 954 of base ring component 950, first longitudinal portion 954 of base ring component 950 radially compresses onto the introducer sheath. Stated another way, due to the internal taper of removable ring component 962, the flexible material of first longitudinal portion 954 is tightened or clasped onto the introducer sheath as first longitudinal portion 954 passes up through or is threaded into opening 930 of removable ring component 962 to releasably secure or lock suture collar 900 to the introducer sheath. Removable ring component 962 is thus tightly disposed over or locked onto base ring component 950, which is tightened or locked onto the outer surface of the introducer sheath at a desired longitudinal position, and suture collar 900 is not able to slide or move freely over the outer surface of the introducer sheath.

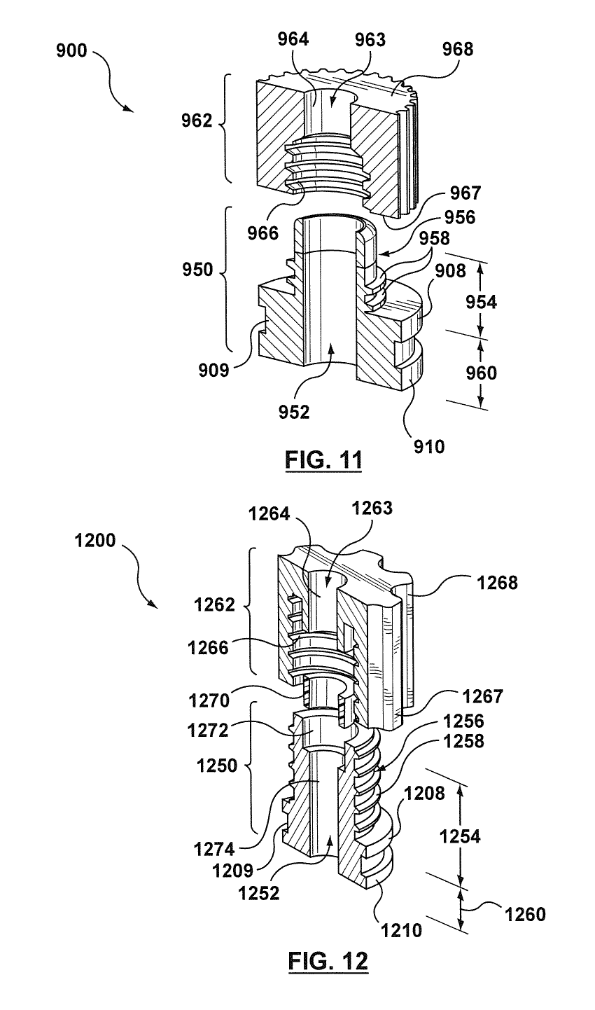

FIG. 12 illustrates a sectional perspective exploded view of another embodiment of an adjustable suture collar 1200 that is configured to be slidingly disposed over an introducer sheath (not shown in FIG. 12) and is configured to be selectively tightened or locked into place. Adjustable suture collar 1200 includes three main components including a base ring component 1250, a removable ring component 1262, and a gasket 1270. Base ring component 1250 is similar to base ring component 950 and defines an opening 1252 there-through that is configured to receive an introducer sheath (not shown in FIG. 12). Base ring component 1250 has a first longitudinal portion 1254 having an outer surface 1256 with a plurality of threads 1258 formed thereon and a second longitudinal portion 1260. Second longitudinal portion 1260 includes a first annular ridge or flange 1208 at a first end or edge thereof and a second annular ridge or flange 1210 at a second or opposing end or edge thereof. Each flange 1208, 1210 extends radially outward such that the outer diameter of the outer ring component at each flange is greater than the outer diameter of the outer ring component at an intermediate portion 1209. Each flange 1208, 1210 also extends around the full circumference of outer ring component 1202.

Removable ring component 1262 is similar to removable ring component 962 and defines an opening 1263 there-through and having an inner surface 1264 with a plurality of grooves 1266 that are configured to mate with the plurality of threads 1258 on first longitudinal portion 1254 of base ring component 1250. A diameter or size of inner surface 1264 tapers or decreases from a first end 1267 to a second end 1268, and is sized such that at least a portion of inner surface 1264, i.e. second end 1268, has a smaller diameter than an outer diameter of first longitudinal portion 1254 of base ring component 1250. First and second longitudinal portions 1254, 1260 of base ring component 1250 and removable ring component 1262 are formed from a relatively rigid, non-compressible material.

First longitudinal portion 1260 of base ring component 1250 includes an annular groove 1272 formed on an inner surface 1274 thereof. Annular groove 1272 is sized to receive gasket 1270 that is formed from a flexible, compressible material. Gasket 1270 may be a removable component that is positioned within annular groove 1272, or may be molded into annular groove 1272 so as to not be removable therefrom. In order to be tightened or locked onto the outer surface of the introducer sheath, i.e., in order to change or transform suture collar 1200 from its first configuration into its second configuration, removable ring component 1262 is screwed onto base ring component 1250 via mating grooves 1266 and threads 1258 of removable ring component 1262 and base ring component 1250, respectively. When removable ring component 1262 is screwed onto base ring component 1250 and removable ring component 1262 is concentrically disposed over first longitudinal portion 1254 of base ring component 1250, gasket 1270 radially compresses onto the introducer sheath. Stated another way, due to the internal taper of removable ring component 1262, the flexible material of gasket 1270 is tightened or clasped onto the introducer sheath as first longitudinal portion 1254 passes up through or is threaded into opening 1230 of removable ring component 1262 to releasably secure or lock suture collar 1200 to the introducer sheath. Removable ring component 1262 is thus tightly disposed over or locked onto base ring component 1250, which is tightened or locked onto the outer surface of the introducer sheath at a desired longitudinal position via gasket 1270, and suture collar 1200 is not able to slide or move freely over the outer surface of the introducer sheath. Removable ring component 1262 may be screwed or rotated in an opposite direction to loosen the removable ring component relative to the introducer sheath so that the longitudinal position of suture collar 1200 may be slidingly readjusted as desired.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. A suture collar configured for use with an introducer sheath, the suture collar comprising:
a single structure outer ring component defining an opening there-though, the single structure outer ring component being configured to be sutured to a target surface of a patient; and
an inner ring component concentrically disposed within the opening of the single structure outer ring component such that in a first configuration of the suture collar a longitudinal axis of the inner ring component is aligned with a longitudinal axis of the single structure outer ring component, the inner ring component defining an opening there-through that is configured to receive the introducer sheath,
wherein an inner surface of the single structure outer ring component is concave, and an outer surface of the inner ring component is convex and is configured to slidingly mate with the concave inner surface of the single structure outer ring component to permit the inner ring component to move relative to the single structure outer ring component
wherein the inner ring component is configured to move relative to the single structure outer ring component such that in a second configuration of the suture collar the longitudinal axis of the inner ring component is angularly offset with the longitudinal axis of the single structure outer ring component.

2. The suture collar of claim 1, wherein the single structure outer ring component includes a flange having a plurality of suture holes formed there-through such that the single structure outer ring component is configured to be sutured to a target surface of a patient.

3. The suture collar of claim 2, wherein the single structure outer ring component has first and second opposing ends and the flange is formed at the second end of the single structure outer ring component.

4. The suture collar of claim 1, wherein the opening of the inner ring component is sized to slidingly receive the introducer sheath and the opening of the inner ring component is also sized such that movement of one of the inner ring component or the introducer sheath results in movement of the other of the inner ring component or the introducer sheath.

5-8. (canceled)

9. An introducer assembly comprising:
an introducer sheath; and
a suture collar configured to be positioned over the introducer sheath, the suture collar including
a single structure outer ring component defining an opening there-though, the single structure outer ring component being configured to be sutured to a target surface of a patient, wherein an inner surface of the single structure outer ring component is concave, and
an inner ring component concentrically disposed within the opening of the single structure outer ring component such that in a first configuration of the suture collar a longitudinal axis of the inner ring component is aligned with a longitudinal axis of the single structure outer ring component, the inner ring component defining an opening there-through that is configured to receive the introducer sheath,
wherein an outer surface of the inner ring component is convex and is configured to slidingly mate with the concave inner surface of the single structure outer ring component to permit the inner ring component and the introducer sheath received therein to move relative to the single structure outer ring component such that in a second configuration of the suture collar the longitudinal axes of the inner ring component and the introducer sheath received therein are angularly offset with the longitudinal axis of the single structure outer ring component.

10. The introducer assembly of claim 9, wherein the single structure outer ring component includes a flange having a plurality of suture holes formed there-through such that the single structure outer ring component is configured to be sutured to a target surface of a patient.

11. The introducer assembly of claim 10, wherein the single structure outer ring component has first and second opposing ends and the flange is formed at the second end of the single structure outer ring component.

12. The introducer assembly of claim 9, wherein the introducer sheath is sized to slidingly receive a prosthetic valve delivery system.

13. The introducer assembly of claim 9, wherein the opening of the inner ring component is sized to slidingly receive the introducer sheath and the opening of the inner ring component is also sized such that movement of one of the inner ring component or the introducer sheath results in movement of the other of the inner ring component or the introducer sheath.

14-20. (canceled)
